(19) United States
(12) Patent Application Publication
(10) Pub. No.: US 2008/0125869 A1
(43) Pub. Date: May 29, 2008

(54) Surgical Grafts

(76) Inventors: Adrian Paz, Petach Tikva (IL); Gilad Heftman, Kibbutz Ein Gev (IL)

Correspondence Address: NATH & ASSOCIATES, 112 South West Street, Alexandria, VA, 22314, US

(21) Appl. No.: 11/667,189

(22) PCT Filed: Nov. 8, 2005
(86) PCT No.: PCT/IL05/01167

§ 371 Date: Jan. 14, 2008

Related U.S. Application Data
(60) Provisional application No. 60/625,725, filed on Nov. 8, 2004.
(60) Provisional application No. 60/696,516, filed on Jul. 6, 2005.

(51) Int. Cl. A61F 2/02

(52) U.S. Cl. 623/23.72; 600/30

(57) ABSTRACT

Provided is a surgical graft having one or more pockets adapted to receive a shaft tip of a surgical fastening device. The graft of the invention may be made from a biodegradable material, a biological material, a non-biodegradable material, biodegradable material, or a combination of any of these. The graft may be of a mesh construction, and may have an elongated shape, or may be shaped as a patch. The graft may be used, for example, in a method for treating urinary incontinence, vaginal vault repair, posterior vaginal wall prolapse, anterior vaginal wall prolapse and inguinal hernia. The invention also provides a system including a surgical graft of the invention, one or more surgical fasteners, and a surgical fastening device having a shaft and configured to eject at least one of the surgical fasteners from the tip of the shaft.

### FIELD OF THE INVENTION

This invention relates to surgical grafts for attachment to a body tissue.

### BACKGROUND OF THE INVENTION

Surgical fasteners are used instead of surgical suturing, which is often both time consuming and inconvenient, in order to join two tissue locations. A surgeon can often use a stapling apparatus to implant a fastener into a body tissue and thus accomplish in a few seconds, what would take a much longer time to suture. A surgical fastener is used, for example in inguinal hernia surgery to fasten polypropylene mesh to the abdominal wall in order to reinforce the abdominal wall.

A surgical fastening device is used to insert a surgical fastener into a body tissue. In these devices, one or more surgical fasteners are contained within a cartridge that are sequentially deployed by an activating mechanism contained in the fastening device. When the body tissue into which a fastener is to be inserted is accessible from only one direction, a fastening device is usually used having a slender shaft. Deployment of a fastener by these devices involves bringing the tip of the shaft to a tissue site and ejecting a fastener from the tip of the shaft. The fastener may become affixed to the tissue site, for example, by undergoing a deformation as it inserts into the tissue, or by rotating as it is ejected so as to screw into the tissue. Surgical fastening devices having a slender shaft are disclosed in U.S. Pat. Nos. 5,582,616, 5,810,882, 5,830,221, 5,470,010, 5,582,616, and in WO 2005/0044727. These systems may be used, for example, in inguinal hernia surgery to fasten a polypropylene mesh to the abdominal wall in order to reinforce the abdominal wall.

### SUMMARY OF THE INVENTION

In its first aspect, the present invention provides a surgical graft for attachment at two or more tissue locations and for reinforcing tissues. The surgical graft of the invention comprises one or more blind sacs or pockets dimensioned to receive the distal end of a surgical fastening device. In use, the tip of the shaft of a surgical fastening device is inserted into a pocket of the graft which is then brought to a tissue site. A surgical fastener is then ejected from the tip of the shaft so as to attach the pocket to the tissue site. The graft of the invention may be formed from a continuous material or may be constructed as a mesh.

The graft may be made of a non-biodegradable material such as polypropylene, a biodegradable material such as PLA, PLGA, polycaprolactone or other such biocompatible materials; from biological materials containing collagen fibers, or any combination of such materials.

In one embodiment of the invention, the graft is elongated in shape having a pocket at each end. In this embodiment each end of the graft can be pinned to a different tissue site, so as to join two tissue sites by the graft. The graft may be formed from a hollow cylinder of material that is closed at a first end into a first pocket and is closed at a second end into a second pocket. The wall of the cylinder may have one or more openings so as to allow the tip of a shaft of a surgical fastening device to be inserted into the interior of the hollow cylinder and to be introduced into any one of the pockets. In another embodiment of the invention, the graft is in the form of a patch or sling, with one or more pockets being located on the periphery of the graft. This allows the graft to be attached to any number of tissue sites simultaneously.

Thus, in its first aspect, the invention provides a surgical graft having one or more pockets adapted to receive a shaft tip of a surgical fastening device.

In its second aspect, the invention provides use of the surgical graft of the invention in a method for treating urinary incontinence, vaginal vault repair, posterior vaginal wall prolapse, anterior vaginal wall prolapse and inguinal hernia.

In its third aspect, the invention provides a systems comprising:
a surgical graft of the invention;
one or more surgical fasteners; and
a surgical fastening device having a shaft and configured to eject at least one of the surgical fasteners from the tip of the shaft.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows an elongated surgical graft in accordance with one embodiment of the invention having two openings;

FIG. 2 shows an elongated surgical graft in accordance with another embodiment of the invention having one opening;

FIG. 3 shows an elongated surgical graft in accordance with another embodiment of the invention having two openings;

FIG. 4 shows repair of stress incontinence using a surgical fastening device of the invention;

FIG. 5 shows an elongated surgical graft in accordance with another embodiment of the invention;

FIG. 6 shows a system for pinning a surgical filament to body tissues in accordance with the invention.

FIG. 7 shows a surgical graft in accordance with another embodiment of the invention having a rectangular shape;

FIG. 8 shows a surgical graft in accordance with another embodiment of the invention having a rectangular shape; and

FIG. 9 shows a surgical graft in accordance with another embodiment of the invention.

### DETAILED DESCRIPTION OF THE INVENTION

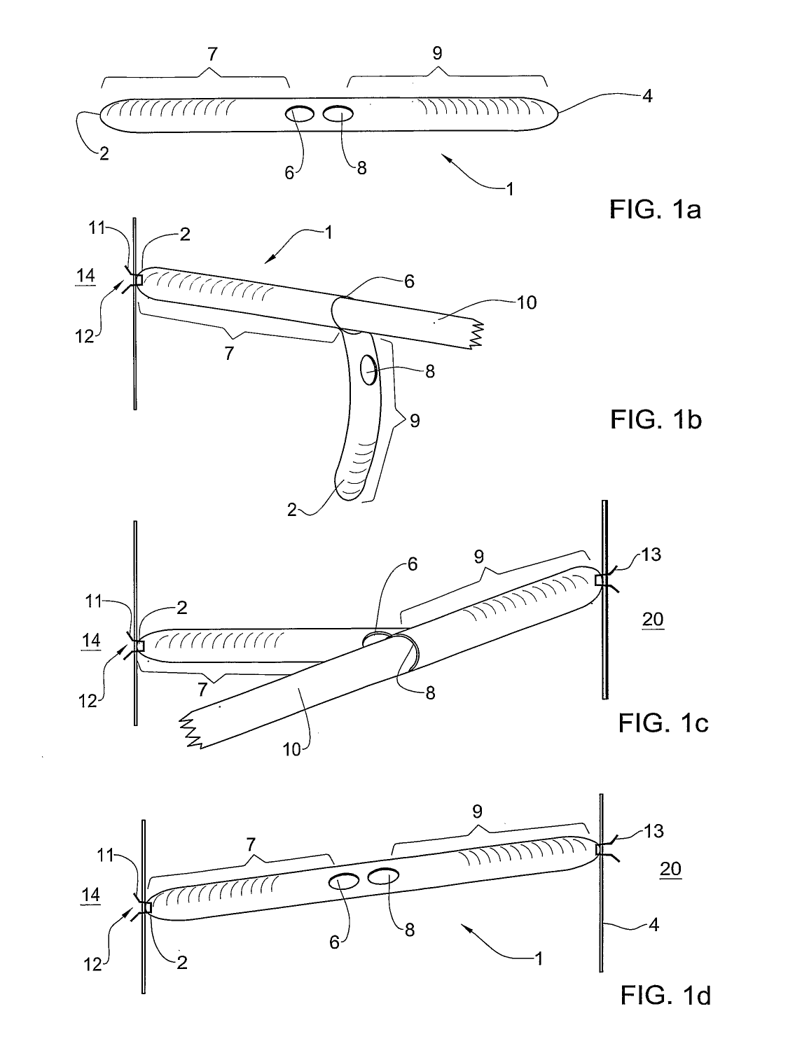

FIG. 1a shows a surgical graft 1 in accordance with one embodiment of this aspect of the invention. The graft 1 is elongated in shape and is formed as a hollow cylinder that is closed at a first end 2 and at a second end 4. The wall of the cylinder may be continuous or may be a mesh. The graft 1 has a first opening 6 and a second opening 8 that are dimensioned to receive the tip of the shaft of a surgical fastening device so as to allow the shaft tip to enter the interior of the hollow cylindrical graft 1, as explained below. The first and second openings 6 and 8 define first and second pockets 7 and 9, respectively, inside the graft 1. The first pocket 7 extends from the first opening 6 to the first end 2. The second pocket 9 extends from the second opening 8 to the second end 4. FIG. 1b shows the graft 1 after the tip 10 of the shaft of a surgical fastening device has been introduced into the interior of the graft through the first hole 6. The shaft tip 10 has been brought to the first pocket 7 of the graft 1. The shaft tip 10 is then brought to a first tissue site 12 of a body tissue 14. In this configuration, a first surgical fastener 11 is ejected from the shaft tip 10 so as to attach the first pocket 7 of the graft 1 to the first tissue site 12 of the body tissue 14.

As shown in FIG. 1c, after the first surgical fastener 11 has been ejected from the shaft tip 10, the shaft tip 10 is removed from the interior of the first pocket 7 of the graft 1 leaving the graft 1 attached to the tissue 14 at the first site 12. At this point, the shaft tip 10 is inserted through the second opening 8 into the second pocket 9. The shaft tip 10 is then brought to a second tissue site 18 in a body tissue 20. In this configuration a second surgical fastener 13 is ejected from the shaft tip 10, so as to attach the second pocket 9 of the graft 1 to the second tissue site. The shaft 10 is then removed from the second pocket 9 leaving the first and second ends 2 and 4, respectively, attached to the first and second locations 12 and 18, respectively, as shown in FIG. 1d.

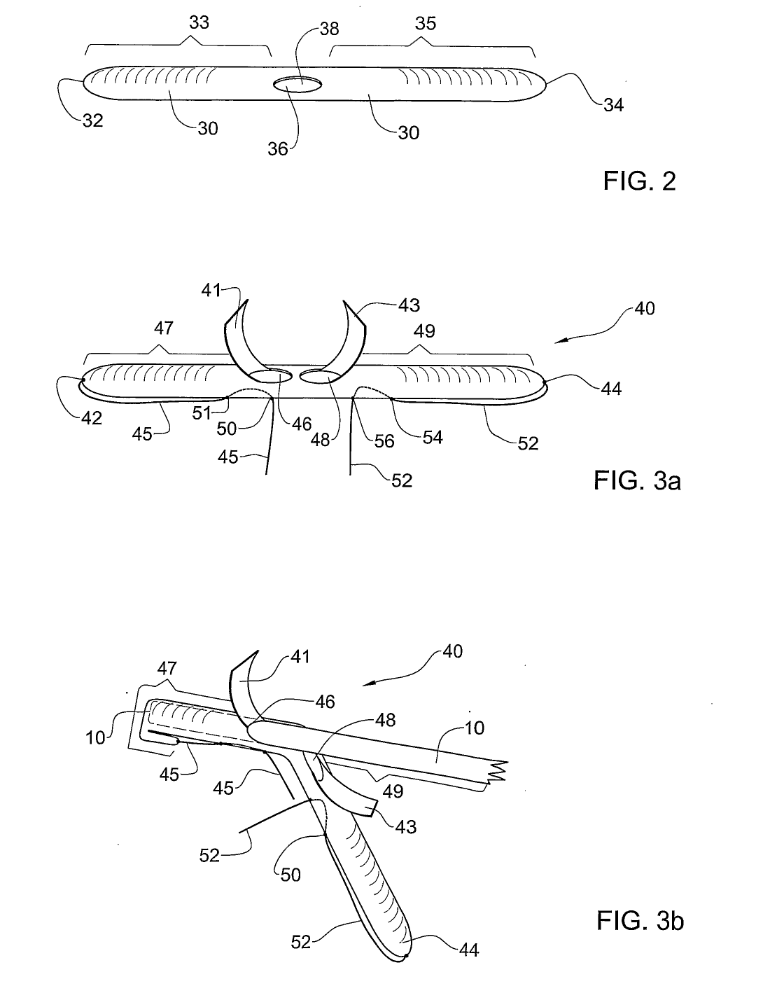

FIG. 2 shows a surgical graft 30 in accordance with another embodiment of the invention. The graft 30 is elongated in shape and is formed as a hollow cylinder that is closed at a first end 32 and at a second end 34. The wall of the cylinder may be continuous or a mesh. The graft 30 has a single opening 36, leading to the interior 38 of the graft 30. The opening 36 defines a first pocket 33 extending from the opening 36 to the first end 32, and a second pocket 35 extending from the opening 36 to the second end 34. In the embodiment shown in FIG. 2, the opening 36 is used to insert the distal end of the shaft of a surgical fastening device into the first pocket when a surgical fastener is to be ejected at the first end 32 as well as to insert the distal end of the shaft into the second pocket when a surgical fastener is to be ejected at the second end 34, as explained above in reference to FIG. 1.

A surgical graft 40 in accordance with another embodiment of the invention is shown in FIG. 3a. The graft 40 is provided with a first flap 41 extending from a first pocket 47 adjacent to the first opening 46. The graft 40 is also provided with a second flap 43 extending from the second pocket 49 adjacent to a second opening 48. As explained below, the flaps 41 and 43 aid in inserting the shaft tip into the pockets 47 and 49. The graft 40 is further provided with a first string 45 attached to the first end 42 of the graft 40. The first string 45 extends from the first end 42, along the outside of the first pocket 47, then passes through the wall of the graft 40 at a location 51 into the interior of the first pocket and then passes through the wall of the graft at a location 50 to the exterior of the graft. Similarly, the graft 40 is provided with a second string 52 attached to the second end 44 of the graft 40. The second string 52 extends from the second end 44 along the outside of the second pocket 49, then passes through the wall of the graft 40 at a location 54 into the interior of the second pocket, and then passes through the wall of the graft at a location 56 to the exterior of the filament. As explained below, the first and second strings 45 and 52 allow the graft 40 to be tautly attached at its first and second pockets to body tissue sites.

FIG. 3b shows the graft 40 after the shaft tip 10 of a surgical fastening device has been introduced into the interior of the first pocket 47 through the first opening 46. The shaft tip 10 has been brought to a predetermined position in the first pocket 47 and the first pocket is folded over the shaft tip 10 by pulling on the first string 45. This allows a first surgical fastener to be ejected from the shaft tip at any desired position in the first pocket. The first flap may be pulled so as to assist in the insertion of the shaft tip 10 into the first pocket. After the first fastener has been ejected, the shaft tip 10 is removed from the first pocket 47 and inserted into the second pocket 49. The process is repeated and a second fastener is ejected from the shaft tip at a desired tissue site in the second pocket. In this way, the distance between the first and second fasteners in the graft 40 can be controlled so as to ensure that the graft is stretched tautly between the first and second locations.

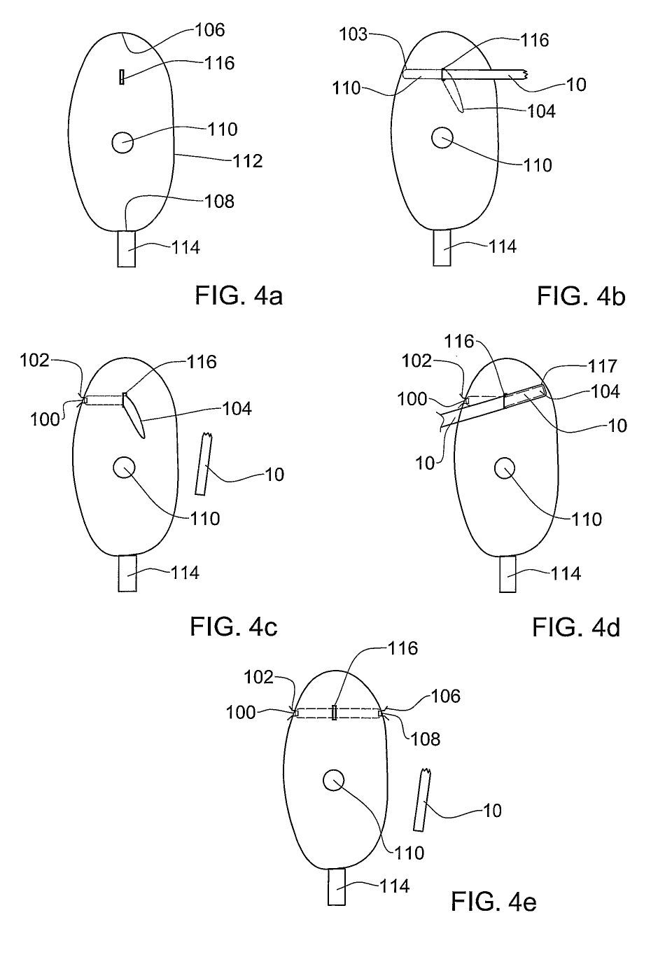

FIG. 4 shows use of a surgical graft 104 of the invention, such as any one of the grafts 1, 30, and 40, in a method of vaginal repair of stress incontinence. The patient is in lithotomy position with the legs supported by stirrups. The vaginal introitus is exposed. Local anesthesia of the planned incision and dissection path is performed and the anterior vaginal wall is hydro-dissected from the urethra and overlaying tissue. An incision is performed on the anterior vaginal wall 1 cm proximal to the urethral meatus. The vaginal wall is dissected laterally by sharp and blunt dissection to the lateral pelvic wall keeping bellow the endopelvic fascia.

The procedure is shown in a vaginal view in FIG. 4, in which the orifice 112 is held open by means of a retractor 114. The anterior vaginal wall 106, the posterior vaginal wall 108 and the cervix 110 are visible in this view. As shown in FIG. 4a, an incision 116, 5 to 10 mm in length, is made on the anterior vaginal wall over the urethra. A plane is then developed bilaterally between the vaginal wall and the urethopelvic ligament toward the attachment of this ligament to the arcus tendineous of the endopelvic fascia. The tip of the shaft is inserted into the interior of the first pocket of the elongated graft. The tip of the shaft with the first end of the elongated graft mounted on it is introduced through the incision 116 towards the side wall of the pelvis (FIG. 4b). The position of the distal end of the shaft in the pocket may be determined using the string 45 and the flap 41, if the graft 40 is used. A surgical fastener 102 is then ejected from the fastening device so as to attach the first end of the graft 104 at a first location 100 to the side wall of the pelvis on one side. The shaft tip 10 is then removed from the incision 116 (FIG. 4c). The shaft is then inserted into the interior of the second pocket of the filament 117. The tip of the shaft with the second end of the elongated graft mounted on it is then reintroduced through the incision 116 to the opposite side wall of the pelvis (FIG. 4d), and a second fastener 106 is ejected from the fastening device so as to attach the second end of the elongated graft at a second location 108 on the second side of the endopelvic fascia. The shaft tip 10 is then removed from the incision (FIG. 4e).

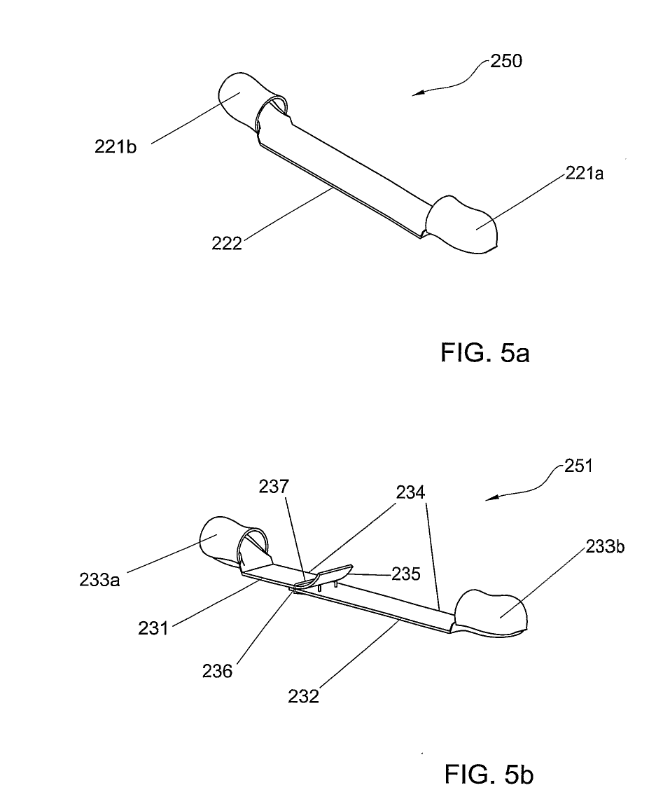

FIG. 5 shows a surgical graft 250 in accordance with another embodiment of the invention. The graft 250 includes a strip 222 that may be, for example, 7 to 15 mm width, and a first pocket 221a and a second pocket 221b at the ends of the strip 222. The length of the strip segment 222 can be adjusted by passing one or more sutures through the strip and folding the strip on itself to fix its length as required in any application. The pockets 221a and 221b, may be, for example, between 1 to 5 cm in length. As shown in FIG. 5b, a graft of the invention 251 may be composed of two or more strip segments 231 and 232 each of which ending with a pocket 233a and 233b, respectively. The strip 231 passes through an opening 236 in the strip 232 so that the length of resulting elongated graft can be adjusted. The two strips 231 and 232 are fixed to one another, for example, by a suture or by a locking pin 237, or other mechanism Alternatively, after attaching the pockets 233a and b with fasteners to tissue sites, the strips can be adjusted to achieve the proper tension and then sutured to one another, and any excess material can be trimmed from the segments 231 and 232.

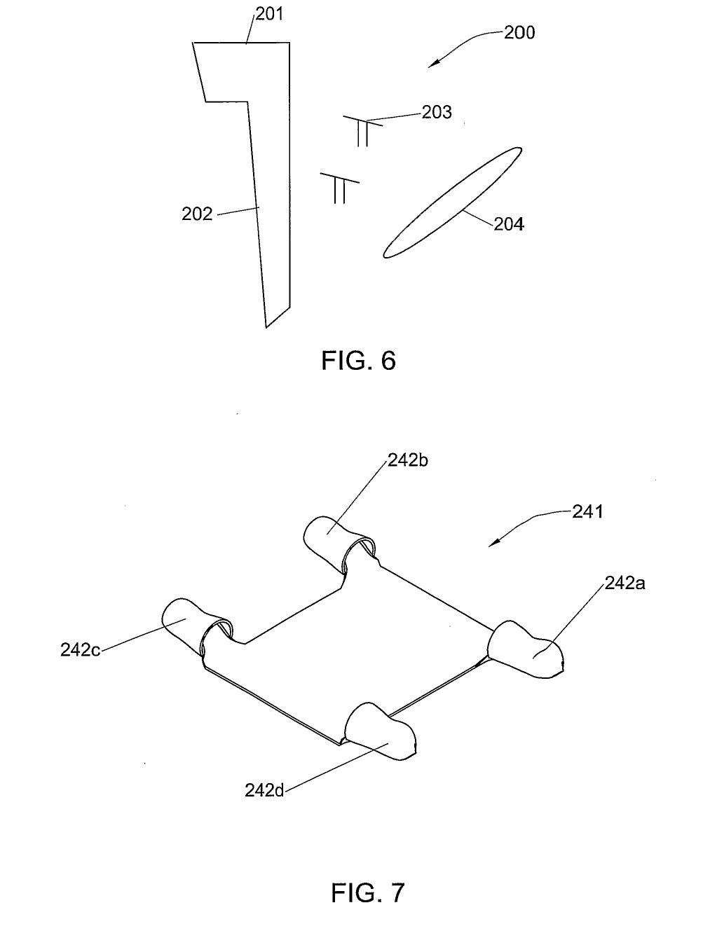

FIG. 6 shows a system 200 for attaching a surgical graft of the invention to body tissues in. The system 200 comprises a surgical fastening device 201 having a slender shaft 202, one or more surgical fasteners 203 and one or more surgical grafts of the invention 204. The fastening device 201 may be any fastening device known in the art having a slender shaft 202 and an activating mechanism that allows the surgical fasteners 203 to be ejected from the tip of the shaft and affixed in a body tissue.

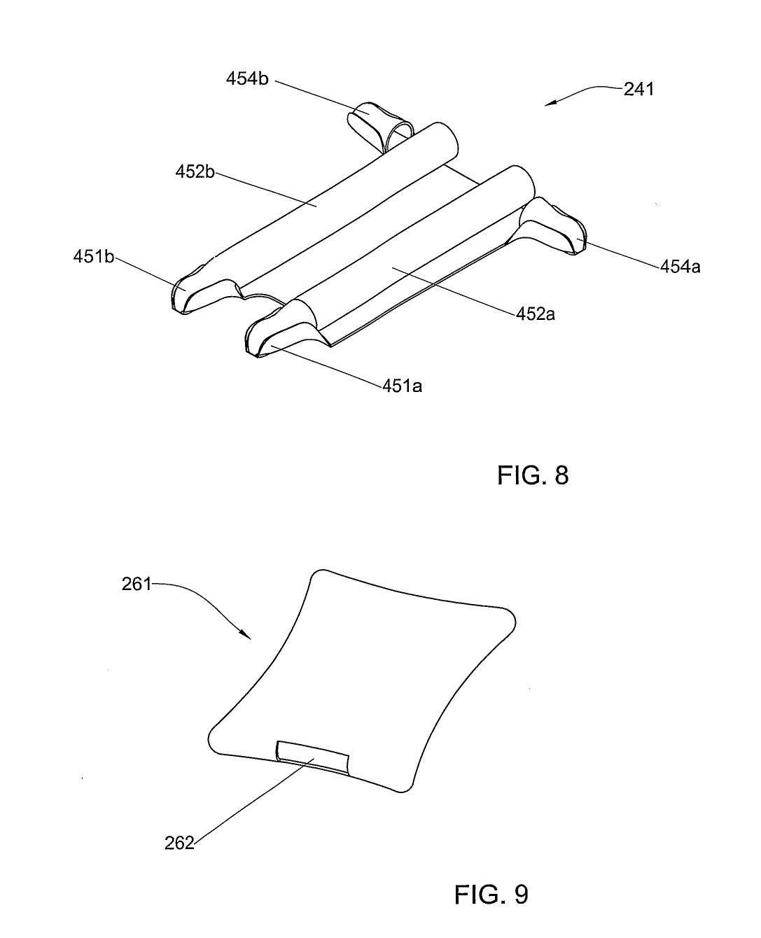

FIG. 7 shows a surgical graft 241 in accordance with another embodiment of the invention. The graft 241 is in the form of as patch, and can be used for supporting or reinforcing a body organ. The graft 241 has a rectangular shape. This is by way of example only, and the graft 241 may have any shape as required in any application. One or more pockets 242 are provided. Four pockets 242a to d are shown in FIG. 6. This is by way of example only, and the graft 241 may be provided with any number of pockets, as required in any application. The size of the graft 241 may be, for example, 4 to 10 cm long and 3 to 7 cm in width. The edges of the graft may be straight lines or they may be curved or notched. The pockets may be attached at corners of the graft or may be attached at some distance from the edge of the graft. In another embodiment shown in FIG. 8, one or more of the pockets 451 are provided with an associated sleeve 452 that facilitates insertion of a shaft tip of a surgical fastening device into the pocket by guiding the shaft tip to the pocket. The sleeve may be detachable from its pocket after deployment of the fastener. One or more additional pockets 454 may be provided not having an associated sleeve.

In another embodiment shown in FIG. 9, a graft 261 is formed as a rectangular sac with an edge provided with an opening 262 for insertion of a shaft of a fastening device.

The graft of the invention may also be used in a method for repairing anterior vaginal wall prolapse; vaginal vault and or posterior vaginal wall prolapse. For repairing posterior vaginal wall prolapse, the patient is in lithotomy position with the legs supported by stirrups. The vaginal introitus is exposed. Local anesthesia of the planned incision and dissection path is performed and the posterior vaginal wall is hydro-dissected from the underlying tissue. A transverse incision, an inverted T shaped incision, or a longitudinal incision is performed between the posterior vaginal wall and the perineum. The vaginal wall is dissected in the middle and laterally from the rectum by sharp and blunt dissection to the lateral pelvic wall and down to the sacrospinous ligament. The shaft of a fastening device is introduced in a pocket of a graft of the invention and the distal end of the shaft with the enveloping pocket is inserted through the opening in the vaginal wall and through the channel created below the vaginal wall, and a fastener is deployed into the tissue near the sacrospinous ligament, or into the tissue at the side wall of the pelvis through the enveloping pocket. Alternatively, the mesh may be introduced through the incision and positioned properly and the distal end of the shaft introduced through the sleeve connected to one of the pockets and a fastener deployed into the tissue near the sacrospinous ligament through the enveloping mesh. Then the fastening device is introduced through the sleeve connected to the second pocket and the procedure is repeated on the opposite side. The mesh may be fixed laterally to tissues near the incision by two additional fasteners through the 2 additional pockets provided with the mesh. At the end of the operation the incision is closed with absorbable sutures.

The graft of the invention may also be used in a method for repairing anterior vaginal wall prolapse. The patient is in lithotomy position with the legs supported by stirrups. The vaginal introitus is exposed. Local anesthesia of the planned incision and dissection path is performed and the anterior vaginal wall is hydro-dissected from the overlaying tissue. A transverse incision, or an inverted T shaped incision, or a longitudinal incision is performed at the bladder neck. The vaginal wall is dissected in the middle and laterally from the urethra and bladder base by sharp and blunt dissection to the lateral pelvic wall and down to the arcus tendineous of endopelvic fascia, or the tissue near the side wall of the pelvis. One of the fastening devices described in previous embodiments is introduced in one pocket of the mesh and the fastening device with the enveloping pocket is inserted through the opening in the vaginal wall and through the channel created above the vaginal wall and the fastener is deployed into the tissue near the arcus tendineous of endopelvic fascia, or the tissue near the side wall of the pelvis through the enveloping mesh. Alternatively, the mesh may be introduced through the incisions and positioned properly and the fastening device is introduced through the sleeve connected to one of the pockets and the fastener deployed into the tissue near the arcus tendineous of endopelvic fascia, or the tissue near the side wall of the pelvis through the enveloping mesh. Then the fastening device is introduced through the sleeve connected to the second pocket and same procedure is performed on the opposite side. The mesh may be fixed laterally to tissues at the lateral pelvic wall by two additional fasteners through the 2 additional pockets provided with the mesh. At the end of the operation the incision is closed with absorbable sutures.

The graft of the invention may be used in a method for repairing an inguinal hernia. The patient lays supine. The skin and subcutaneous tissue overlaying the external ring of the inguinal canal is infiltrated with anesthetic solution. A 2 to 3 cm skin incision is performed over the external ring of the inguinal canal. The inguinal cord with the hernia sac is isolated. The cord is infiltrated with anesthetic solution. The cremaster sheath is opened and the sac is isolated and dissected toward and within the external ring. In case of a voluminous sac the external ring of the inguinal canal is opened to facilitate dissection of the sac to the sac neck in the posterior wall of the inguinal canal—in case of direct hernia—or to the internal ring of the inguinal canal—in case of the indirect hernia. Blunt and sharp dissection is used. In case of a voluminous inguino-scrotal sac, the sac is incised and the distal sac is left attached to the cord elements. A rectangular mesh is used for repair of the weakness of the posterior wall of the inguinal canal. The mesh is attached at the conjoint tendon of the inguinal canal superiorly and at the inguinal ligament inferiorly, at one or more fixation points using the previously described fasteners and fastening devices under direct vision and using palpation. A sac type of mesh may be particularly suited for such an application.

1. A surgical graft having one or more pockets adapted to receive a shaft tip of a surgical fastening device.

2. The surgical graft according to claim 1 having two or more pockets adapted to receive a shaft tip of a surgical fastening device.

3. The surgical graft according to claim 1 made from a biodegradable material.

4. The surgical graft according to claim 1 made from a biological material

5. The surgical graft according to claim 1 made from a combination of non-biodegradable material and biodegradable material.

6. The surgical graft according to claim 1 made from a combination of non-biodegradable material and biological material.

7. The surgical graft according to claim 1 made from a combination of biodegradable material and biological material.

8. The surgical graft according to claim 1 having a mesh construction.

9. The surgical graft according to claim 1 having a elongated shape.

10. The surgical graft according to claim 9 having a pocket at each end of the elongated shape.

11. The surgical graft according to claim 10 wherein comprising a strip joining the pockets.

12. The surgical graft according to claim 11 wherein the strip has an adjustable length.

13. The surgical graft according claim 9 further comprising a first string attached to the first end and a second string attached to the second end.

14. The surgical graft according to claim 13 further comprising a flap adjacent to at least one of the openings.

15. The surgical graft according to claim 1 in the form of a patch.

16. The surgical graft according to claim 1 being rectangular in shape.

17. The surgical graft according to claim 16 having a pocket each of one or more corners of the rectangle.

18. The surgical graft according to claim 16 further comprising at least one sleeve guiding a shaft tip of a surgical fastening device to a pocket.

19. Use of the surgical graft according to any one of the previous claims in a method for treating urinary incontinence, vaginal vault repair, posterior vaginal wall prolapse, anterior vaginal wall prolapse and inguinal hernia.

20. A system comprising:
(a) A surgical graft according to claim 1;
(b) One or more surgical fasteners;
(c) A surgical fastening device having a shaft and configured to eject at least one of the surgical fasteners from the tip of the shaft.
